(19) United States
(12) Patent Application Publication
(10) Pub. No.: US 2025/0265180 A1
(43) Pub. Date: Aug. 21, 2025

(54) SYSTEM FOR LEVERAGING SYNTHETIC DNA FOR COMPUTER STORAGE

(71) Applicant: Bank of America Corporation, Charlotte, NC (US)

(72) Inventors: Maneesh Kumar Sethia, Hyderabad Telangana (IN); Manimaran Sundaravel, Chennai Tamil Nadu (IN); Shailendra Singh, Maharashtra (IN)

(21) Appl. No.: 18/581,531

(22) Filed: Feb. 20, 2024

(51) Int. Cl. G06F 12/02

(52) U.S. Cl. CPC G06F 12/02

(57) ABSTRACT

A system for storing data on deoxyribonucleic acid (“DNA”) may include a receiver, a processor and/or a DNA synthesizer. The receiver may receive data files. The processor may segment the data files into a plurality of data packets. The processor may randomly select one or more packets from the plurality of data packets. The processor may combine the selected packets into an output. The processor may attach a random seed to the output. The processor may derive a sequence from the seeded output. The processor may identify the sequence as a valid sequence or a homopolymer. The processor may discard the sequence when the sequence is identified as a homopolymer. The DNA synthesizer may convert the sequence into a DNA quaternary sequence when the sequence is identified as a valid sequence. A DNA quaternary sequence may include DNA bases. The DNA synthesizer may synthesize and store the DNA sequence.

### FIELD OF TECHNOLOGY

Aspects of the disclosure relate to synthetic deoxyribonucleic acid (“DNA”).

### BACKGROUND OF THE DISCLOSURE

Recently, the amount of data generated daily is rapidly increasing. As such, the rapid increase in generated data has created a need for more efficient storage structures.

DNA is a carrier of natural genetic information. As such, DNA provides a stable, resource-efficient, energy-efficient and sustainable storage structure.

It would be desirable to use DNA to store data.

It would be yet further desirable to encode electronic computer sequences on strands of DNA.

### SUMMARY OF THE DISCLOSURE

Systems, apparatus and methods for leveraging synthetic DNA for computer storage may be provided.

Methods may include receiving one or more data files. The data files may include text files, image files, portable document format (“pdf”) files, video files, audio files and any other suitable files.

Methods may include converting the data files binary files. It should be noted that the binary files may encode data using zeros and ones.

Methods may include segmenting the binary file into a plurality of data packets. Methods may include randomly selecting packets from the plurality of data packets. The random selection may include retrieving one, two, three or more packets from the plurality of data packets.

Methods may include combining the selected one or more packets into an output. The combining may utilize an algorithm. The algorithm may be used to process the combination. The algorithm may be an exclusive or operation. The algorithm may be a bitwise addition operation. In some embodiments, an exclusive or operation may be referred to as a bitwise addition operation.

Methods may include attaching a four-byte random seed to the output. Attaching the four-byte random seed to the output may form a seeded output. It should be noted that random seeds greater than, or less than, four bytes may be used in certain embodiments.

Methods may include identifying the sequence as a valid sequence or as an invalid sequence. It should be noted that certain sequences, within DNA, may be difficult to process and error-prone. These sequences may be referred to as homopolymers. Homopolymers may be stretches of DNA bases (mono nucleotides) greater than two bases long which occur together. The DNA bases may include adenine (“A”), thymine (‘T’), cytosine (‘C’) and guanine (‘G’). For example, a ‘ATCCCGC’ may include a homopolymer. The homopolymer may be base ‘C’ with a length of three. These stretches may cause errors when sequencing DNA. Specifically, DNA sequencing technologies read DNA bases by reconstructing the DNA by referring to a sample. Since the bases used for reconstruction are attached with a fluorophore, upon the addition of each subsequent base, the intensity of emitted fluorescence is recorded. The cumulative intensity increases linearly with the number of bases added. However, when a series (greater than two) of identical bases is added, the linearity may be lost. As such, the sequencer may be unable to, over a threshold level of confidence, distinguish between 3 As and 7 As or 8 Ts and 9 Ts. Therefore, methods may include discarding sequences that include homopolymers. Such sequences may be identified as invalid sequences.

The invalid sequence may be a homopolymer. The invalid sequence may include greater than a threshold number of duplicate bases.

Methods may include converting the sequence into a DNA quaternary sequence. As such, the binary sequence, including zeros and ones, may be converted into a DNA quaternary sequence, including As, Ts, Cs and Gs. The converting may be based on a code table.

Methods may include synthesizing the DNA sequence. Methods may include storing the DNA sequence.

### BRIEF DESCRIPTION OF THE DRAWINGS

The objects and advantages of the invention will be apparent upon consideration of the following detailed description, taken in conjunction with the accompanying drawings, in which like reference characters refer to like parts throughout, and in which:

FIGS. 1A, 1B, 1C, 1D and 1E show illustrative diagrams in accordance with principles of the disclosure;

FIGS. 2A, 2B and 2C shows an illustrative listing in accordance with principles of the disclosure; and

FIG. 3 shows an illustrative hybrid diagram/flow chart in accordance with principles of the disclosure.

### DETAILED DESCRIPTION OF THE DISCLOSURE

Apparatus, systems and methods for storing data on DNA is provided. The system may include a receiver operable to receive one or more data files.

The system may include a processing element. The processing element may be operable to segment the one or more data files into a plurality of data packets. The processing element may be operable to randomly select one or more packets from the plurality of data packets. The processing element may be operable to combine the selected one or more packets into an output. The processing element may use an algorithm to combine the selected one or more packets. The algorithm may be an exclusive or operation. The algorithm may be a bitwise addition operation.

The processing element may attach a four-byte random seed to the output. The processing element may derive a sequence from the seeded output. The processing element may identify the sequence as a valid sequence or as an invalid sequence. The invalid sequence may be a homopolymer. The invalid sequence may include greater than a threshold number of duplicate bases. The threshold number may be two, three or any other suitable number. The processing element may discard the sequence when the sequence is identified as an invalid sequence.

The system may include a DNA synthesizer. The DNA synthesizer may, when the sequence is identified as a valid sequence, convert the sequence into a DNA quaternary sequence. The DNA synthesizer may synthesize the DNA sequence. The DNA synthesizer may store the DNA sequence.

Converting the sequence into a DNA quaternary sequence may be based on a code table. The code table may be included as table A.

TABLE A

| Quaternary | Decode |
|---|---|
| Code | Equivalent |
| ACGA | 0 |
| CCGA | 1 |
| GCGA | 2 |
| TCGA | 3 |
| ACTA | 4 |
| CCTA | 5 |
| GCTA | 6 |
| TCTA | 7 |
| ACAA | 8 |
| CCAA | 9 |
| GCAA | 10 |
| TCAA | 11 |
| ACGC | 12 |
| CCGC | 13 |
| GCGC | 14 |
| TCGC | 15 |
| ACTC | 16 |
| CCTC | 17 |
| GCTC | 18 |
| TCTC | 19 |
| ACAC | 20 |
| CCAC | 21 |
| GCAC | 22 |
| TCAC | 23 |
| ACTG | 24 |
| CCTG | 25 |
| GCTG | 26 |
| TCTG | 27 |
| ACAG | 28 |
| CCAG | 29 |
| GCAG | 30 |
| TCAG | 31 |
| ACGG | 32 |
| CCGG | 33 |
| GCGG | 34 |
| TCGG | 35 |
| ACGT | 36 |
| CCGT | 37 |
| GCGT | 38 |
| TCGT | 39 |
| ACTT | 40 |
| CCTT | 41 |
| GCTT | 42 |
| TCTT | 43 |
| ACAT | 44 |
| CCAT | 45 |
| GCAT | 46 |
| TCAT | 47 |
| AGTA | 48 |
| CGTA | 49 |
| GGTA | 50 |
| TGTA | 51 |
| AGAA | 52 |
| CGAA | 53 |
| GGAA | 54 |
| TGAA | 55 |
| AGCA | 56 |
| CGCA | 57 |
| GGCA | 58 |
| TGCA | 59 |
| AGTC | 60 |
| CGTC | 61 |
| GGTC | 62 |
| TGTC | 63 |
| AGAC | 64 |
| CGAC | 65 |
| GGAC | 66 |
| TGAC | 67 |
| AGCC | 68 |
| CGCC | 69 |
| GGCC | 70 |
| TGCC | 71 |
| AGTG | 72 |
| CGTG | 73 |
| GGTG | 74 |
| TGTG | 75 |
| AGAG | 76 |
| CGAG | 77 |
| GGAG | 78 |
| TGAG | 79 |
| AGCG | 80 |
| CGCG | 81 |
| GGCG | 82 |
| TGCG | 83 |
| AGTT | 84 |
| CGTT | 85 |
| GGTT | 86 |
| TGTT | 87 |
| AGAT | 88 |
| CGAT | 89 |
| GGAT | 90 |
| TGAT | 91 |
| AGCT | 92 |
| CGCT | 93 |
| GGCT | 94 |
| TGCT | 95 |
| ATGA | 96 |
| CTGA | 97 |
| GTGA | 98 |
| TTGA | 99 |
| ATAA | 100 |
| CTAA | 101 |
| GTAA | 102 |
| TTAA | 103 |
| ATCA | 104 |
| CTCA | 105 |
| GTCA | 106 |
| TTCA | 107 |
| ATGC | 108 |
| CTGC | 109 |
| GTGC | 110 |
| TTGC | 111 |
| ATAC | 112 |
| CTAC | 113 |
| GTAC | 114 |
| TTAC | 115 |
| ATCC | 116 |
| CTCC | 117 |
| GTCC | 118 |
| TTCC | 119 |
| ATGG | 120 |
| CTGG | 121 |
| GTGG | 122 |
| TTGG | 123 |
| ATAG | 124 |
| CTAG | 125 |
| GTAG | 126 |
| TTAG | 127 |
| ATCG | 128 |
| CTCG | 129 |
| GTCG | 130 |
| TTCG | 131 |
| ATGT | 132 |
| CTGT | 133 |
| GTGT | 134 |
| TTGT | 135 |
| ATAT | 136 |
| CTAT | 137 |
| GTAT | 138 |
| TTAT | 139 |
| ATCT | 140 |
| CTCT | 141 |
| GTCT | 142 |
| TTCT | 143 |
| AAGA | 144 |
| CAGA | 145 |
| GAGA | 146 |
| TAGA | 147 |
| AATA | 148 |
| CATA | 149 |
| GATA | 150 |
| TATA | 151 |
| AACA | 152 |
| CACA | 153 |
| GACA | 154 |
| TACA | 155 |
| AAGC | 156 |
| CAGC | 157 |
| GAGC | 158 |
| TAGC | 159 |
| AATC | 160 |
| CATC | 161 |
| GATC | 162 |
| TATC | 163 |
| AACC | 164 |
| CACC | 165 |
| GACC | 166 |
| TACC | 167 |
| AAGG | 168 |
| CAGG | 169 |
| GAGG | 170 |
| TAGG | 171 |
| AATG | 172 |
| CATG | 173 |
| GATG | 174 |
| TATG | 175 |
| AACG | 176 |
| CACG | 177 |
| GACG | 178 |
| TACG | 179 |
| AAGT | 180 |
| CAGT | 181 |
| GAGT | 182 |
| TAGT | 183 |
| AATT | 184 |
| CATT | 185 |
| GATT | 186 |
| TATT | 187 |
| AACT | 188 |
| CACT | 189 |
| GACT | 190 |
| TACT | 191 |

Apparatus and methods described herein are illustrative. Apparatus and methods in accordance with this disclosure will now be described in connection with the figures, which form a part hereof. The figures show illustrative features of apparatus and method steps in accordance with the principles of this disclosure. It is to be understood that other embodiments may be utilized and that structural, functional and procedural modifications may be made without departing from the scope and spirit of the present disclosure.

The steps of methods may be performed in an order other than the order shown or described herein. Embodiments may omit steps shown or described in connection with illustrative methods. Embodiments may include steps that are neither shown nor described in connection with illustrative methods.

Illustrative method steps may be combined. For example, an illustrative method may include steps shown in connection with another illustrative method.

Apparatus may omit features shown or described in connection with illustrative apparatus. Embodiments may include features that are neither shown nor described in connection with the illustrative apparatus. Features of illustrative apparatus may be combined. For example, an illustrative embodiment may include features shown in connection with another illustrative embodiment.

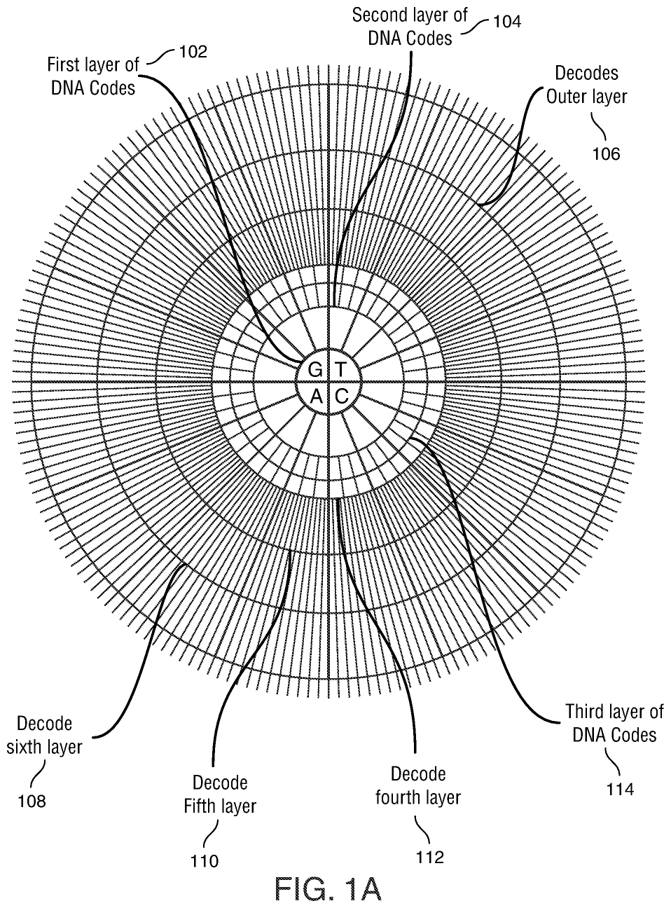

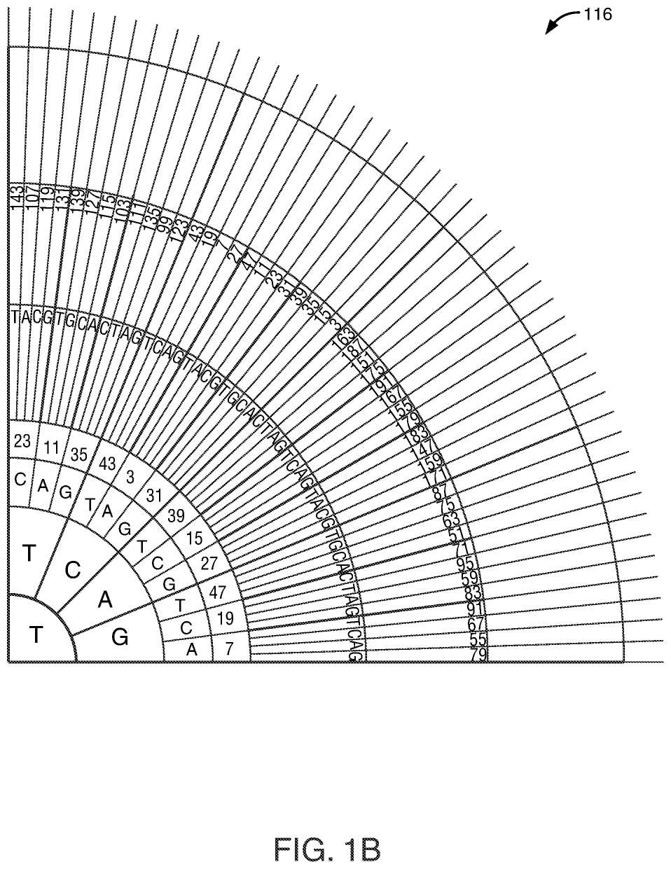

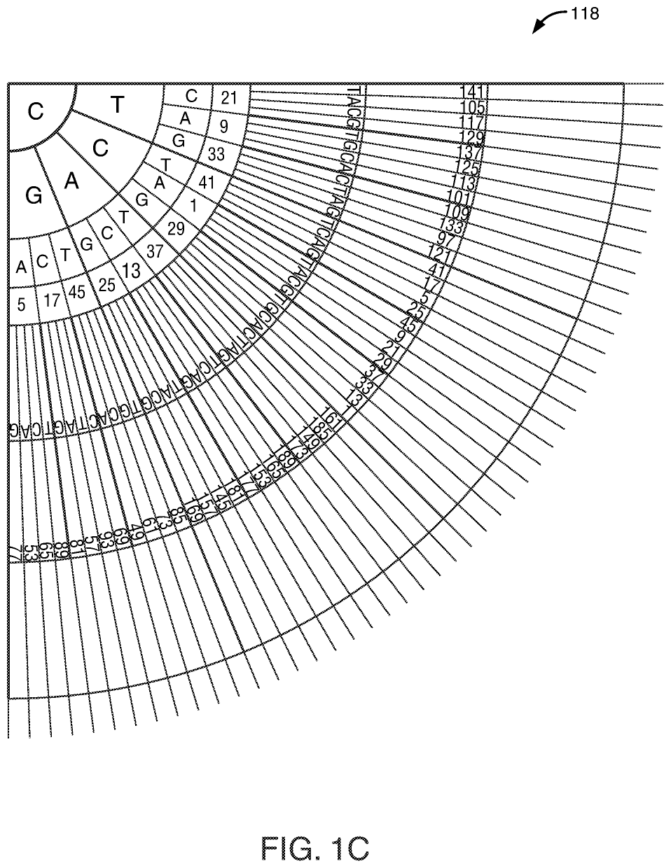

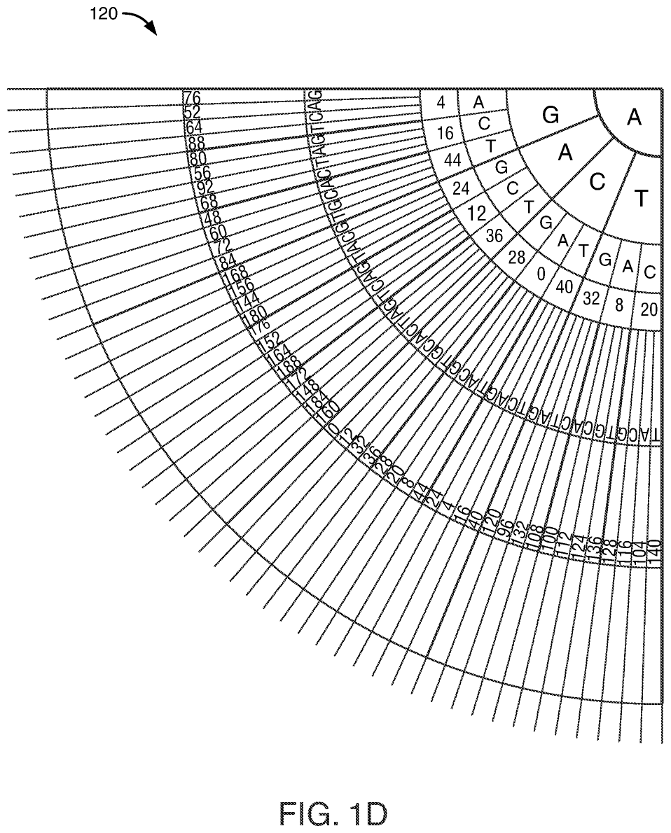

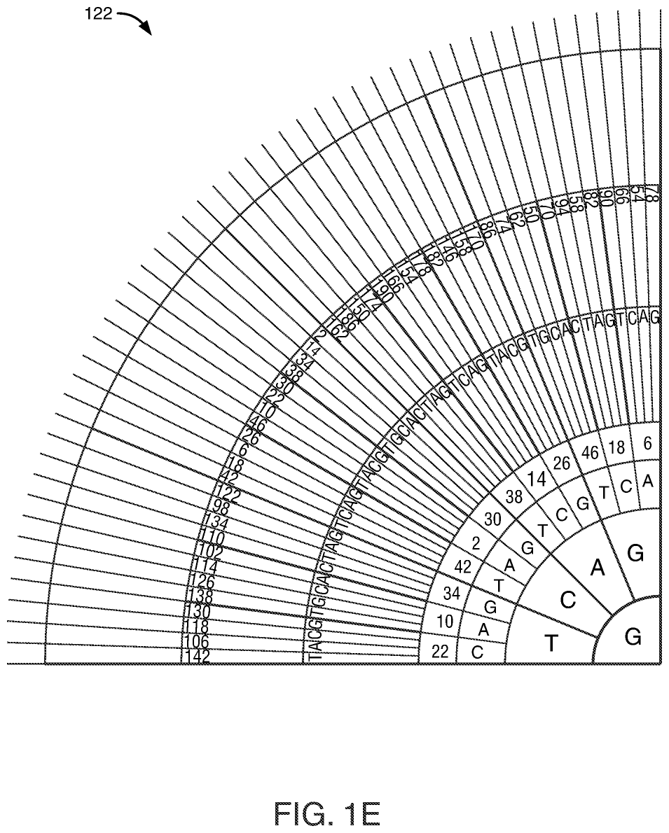

FIGS. 1A, 1B, 1C, 1D, 1E show illustrative diagrams in accordance with principles of the disclosure. FIG. 1A shows an illustrative diagram. The illustrative diagram may be used to convert binary sequences to DNA quaternary codes. The illustrative diagram may also be used to decode DNA sequences to binary numbers.

The illustrative diagram includes multiple layers of DNA codes. The illustrative diagram includes binary (numerical) equivalents.

The first layer of DNA codes is shown at 102. The first layer of DNA codes may include four DNA bases (A, T, C and G). The first layer of DNA codes may correspond to the first digit in a four-digit binary number.

The second layer of DNA codes is shown at 104. The second layer of DNA codes may include an option of selecting one of four DNA bases (A, T, C and G). The second layer of DNA codes may correspond to the second digit in a four-digit binary number.

The third layer of DNA codes is shown at 114. The third layer of DNA codes may include an option for selecting one of three DNA bases (A, T, C and G). The third layer of DNA codes may correspond to third digit in a four-digit binary number. It should be noted that removing the option of one DNA code from the third layer of DNA codes may remove the possibility of creating a homopolymer.

The fourth layer of the diagram, shown at 112, includes a decode layer. The decode layer is a numeric layer. The numbers included in the decode layer may be used to identify a binary number when decoding a sequence created from DNA codes.

The fifth layer of the diagram, shown at 110, may include DNA codes. The fifth layer of the diagram may include an option for selection one of four DNA bases (A, T, C and G). The fifth layer of the DNA codes may correspond to a fourth digit in a four-digit binary number.

The sixth layer of the diagram, shown at 108, may include numerals. The numerals may correspond to a binary equivalent to a four-digit quaternary code. For example, quaternary code CGTA may correspond to numeral 49.

The outer layer of the diagram may be shown at 106.

FIG. 1B shows an illustrative diagram. The illustrative diagram shows quadrant 116. Quadrant 116 may be a detailed section of the diagram shown in FIG. 1A. Quadrant 116 may correspond to quaternary codes that begin with a T.

FIG. 1C shows an illustrative diagram. The illustrative diagram shows quadrant 118. Quadrant 118 may be a detailed section of the diagram shown in FIG. 1A. Quadrant 118 may correspond to quaternary codes that begin with a C.

FIG. 1D shows an illustrative diagram. The illustrative diagram shows quadrant 120. Quadrant 120 may be a detailed section of the diagram shown in FIG. 1A. Quadrant 120 may correspond to quaternary codes that begin with an A.

FIG. 1E shows an illustrative diagram. The illustrative diagram shows quadrant 122. Quadrant 122 may be a detailed section of the diagram shown in FIG. 1A. Quadrant 120 may correspond to quaternary codes that begin with a G.

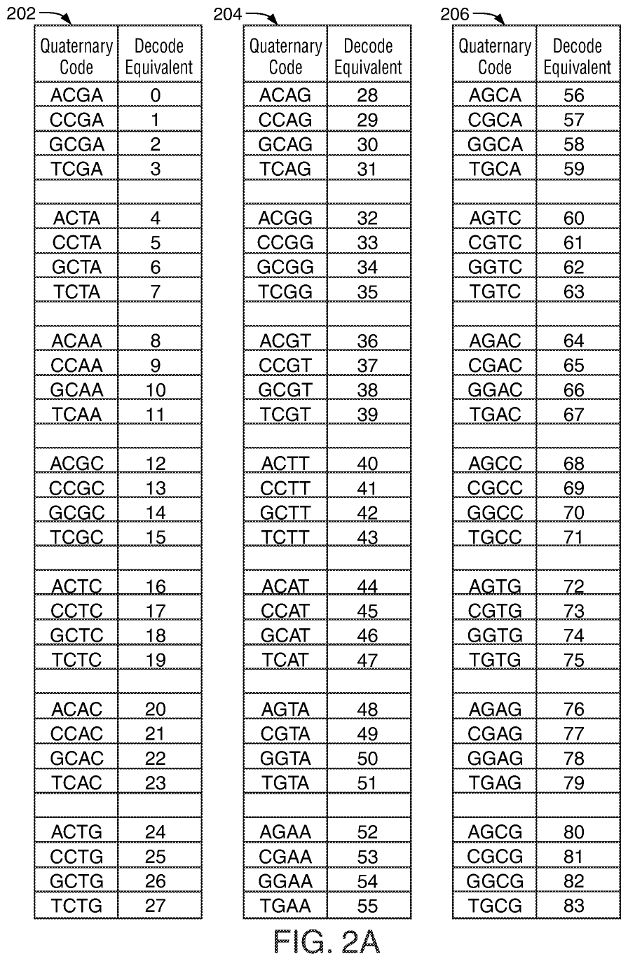

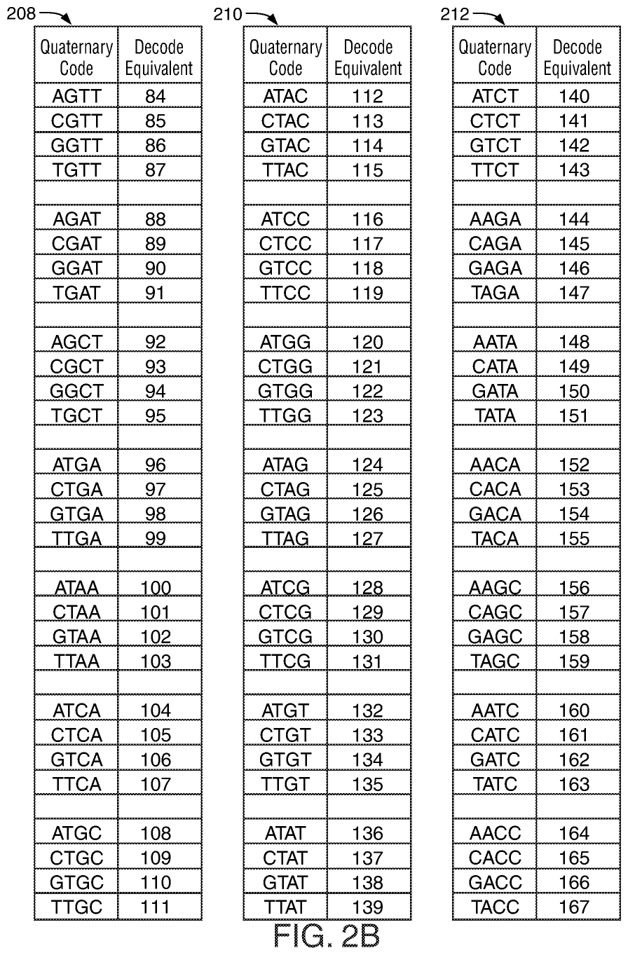

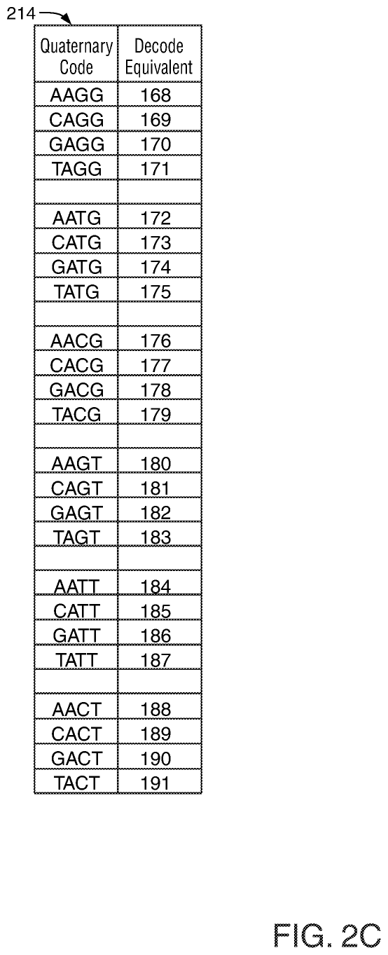

FIGS. 2A, 2B, 2C shows an illustrative listing in accordance with principles of the disclosure.

FIG. 2A shows a first portion of a listing of quaternary codes and decode equivalents. FIG. 2A shows sections 202, 204 and 206. Section 202 shows a listing ranging from numerical decode zero to numerical decode 27. Section 204 shows a listing ranging from numerical decode 28 to numerical decode 55. Section 206 shows a listing ranging from numerical decode 56 to numerical decode 83.

FIG. 2B shows a second portion of the listing of quaternary codes and decode equivalents. FIG. 2B shows sections 208, 210 and 212. Section 208 shows a listing ranging from numerical decode 84 to numerical decode 111. Section 210 shows a listing ranging from numerical decode 112 to numerical decode 139. Section 212 shows a listing ranging from numerical decode 140 to numerical decode 167.

FIG. 2C shows a third portion of the listing of quaternary codes and decode equivalents. FIG. 2C shows section 214. Section 214 shows a listing ranging from numerical decode 168 to numerical decode 191.

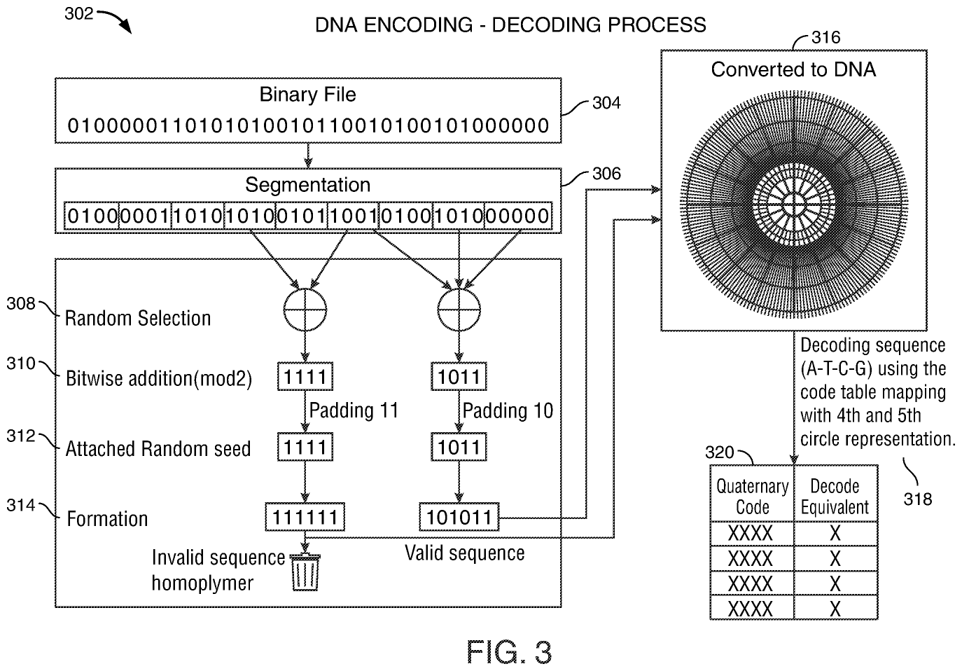

FIG. 3 shows an illustrative hybrid diagram/flow chart in accordance with principles of the disclosure.

The hybrid diagram/flow chart may include DNA encoding/decoding process 302. The process may initiate with receipt of a binary file, shown at 304. A binary file may include one or more zeros and ones.

The process may include segmenting the binary file, as shown at 306. The binary file may be segmented into a plurality of segments. The segments may be the same in length. The segments may be different in length.

The process may include random selection of segments, as shown at 308. One, two or any other suitable number of segments may be selected.

The process may include executing bitwise addition (mod 2) to combine one or more segments, as shown at 310.

The process may include attaching a random seed to each combined segment, as shown at 312.

The process may include forming an output, as shown at 314. The output may include the random seed and the combined segment. The output may identify a binary sequence.

Invalid sequences may be discarded. Invalid sequences may include binary sequences that would generate homopolymers when converted to DNA sequences.

Valid sequences may be converted to DNA sequences using a DNA mapping, as shown at 316. The DNA sequences may be encoded on synthetic DNA. The synthetic DNA may be stored. The stored DNA may be read and decoded at another instance. The stored DNA may be read and decoded using a DNA mapping. The DNA mapping may be the same mapping used to convert the DNA sequence. As such, the 4^{th} and 5^{th} circle representation, indicated at 318, and the code table, shown at 320, may be used to decode stored DNA.

Thus, systems and methods for leveraging synthetic DNA for computer storage are provided. Persons skilled in the art will appreciate that the present invention can be practiced by other than the described embodiments, which are presented for purposes of illustration rather than of limitation. The present invention is limited only by the claims that follow.

What is claimed is:

1. An encoding method for storing data on deoxyribonucleic acid (“DNA”), the method comprising:
receiving one or more data files;
segmenting the one or more data files into a plurality of data packets;
randomly selecting one or more packets from the plurality of data packets;
combining, using an algorithm, the selected one or more packets into an output;
attaching a four-byte random seed to the output;
deriving a sequence from the seeded output;
identifying the sequence as a valid sequence or an invalid sequence;
converting the sequence into a DNA quaternary sequence, said DNA quaternary sequence comprising one or more DNA bases;
synthesizing the DNA sequence; and
storing the DNA sequence.

2. The encoding method of claim 1, wherein the algorithm is an exclusive or operation.

3. The encoding method of claim 1, wherein the algorithm is a bitwise addition operation.

4. The encoding method of claim 1, wherein the invalid sequence is a homopolymer.

5. The encoding method of claim 1, wherein the invalid sequence comprises greater than a threshold number of duplicate bases.

6. The encoding method of claim 1, wherein the one or more DNA bases include adenine, thymine, cytosine and guanine.

7. The encoding method of claim 1, wherein the converting is based on a code table.

8. The encoding method of claim 7 wherein the code table comprises the following code table:

| Quaternary | Decode |
|---|---|
| Code | Equivalent |
| ACGA | 0 |
| CCGA | 1 |
| GCGA | 2 |
| TCGA | 3 |
| ACTA | 4 |
| CCTA | 5 |
| GCTA | 6 |
| TCTA | 7 |
| ACAA | 8 |
| CCAA | 9 |
| GCAA | 10 |
| TCAA | 11 |
| ACGC | 12 |
| CCGC | 13 |
| GCGC | 14 |
| TCGC | 15 |
| ACTC | 16 |
| CCTC | 17 |
| GCTC | 18 |
| TCTC | 19 |
| ACAC | 20 |
| CCAC | 21 |
| GCAC | 22 |
| TCAC | 23 |
| ACTG | 24 |
| CCTG | 25 |
| GCTG | 26 |
| TCTG | 27 |
| ACAG | 28 |
| CCAG | 29 |
| GCAG | 30 |
| TCAG | 31 |
| ACGG | 32 |
| CCGG | 33 |
| GCGG | 34 |
| TCGG | 35 |
| ACGT | 36 |
| CCGT | 37 |
| GCGT | 38 |
| TCGT | 39 |
| ACTT | 40 |
| CCTT | 41 |
| GCTT | 42 |
| TCTT | 43 |
| ACAT | 44 |
| CCAT | 45 |
| GCAT | 46 |
| TCAT | 47 |
| AGTA | 48 |
| CGTA | 49 |
| GGTA | 50 |
| TGTA | 51 |
| AGAA | 52 |
| CGAA | 53 |
| GGAA | 54 |
| TGAA | 55 |
| AGCA | 56 |
| CGCA | 57 |
| GGCA | 58 |
| TGCA | 59 |
| AGTC | 60 |
| CGTC | 61 |
| GGTC | 62 |
| TGTC | 63 |
| AGAC | 64 |
| CGAC | 65 |
| GGAC | 66 |
| TGAC | 67 |
| AGCC | 68 |
| CGCC | 69 |
| GGCC | 70 |
| TGCC | 71 |
| AGTG | 72 |
| CGTG | 73 |
| GGTG | 74 |
| TGTG | 75 |
| AGAG | 76 |
| CGAG | 77 |
| GGAG | 78 |
| TGAG | 79 |
| AGCG | 80 |
| CGCG | 81 |
| GGCG | 82 |
| TGCG | 83 |
| AGTT | 84 |
| CGTT | 85 |
| GGTT | 86 |
| TGTT | 87 |
| AGAT | 88 |
| CGAT | 89 |
| GGAT | 90 |
| TGAT | 91 |
| AGCT | 92 |
| CGCT | 93 |
| GGCT | 94 |
| TGCT | 95 |
| ATGA | 96 |
| CTGA | 97 |
| GTGA | 98 |
| TTGA | 99 |
| ATAA | 100 |
| CTAA | 101 |
| GTAA | 102 |
| TTAA | 103 |
| ATCA | 104 |
| CTCA | 105 |
| GTCA | 106 |
| TTCA | 107 |
| ATGC | 108 |
| CTGC | 109 |
| GTGC | 110 |
| TTGC | 111 |
| ATAC | 112 |
| CTAC | 113 |
| GTAC | 114 |
| TTAC | 115 |
| ATCC | 116 |
| CTCC | 117 |
| GTCC | 118 |
| TTCC | 119 |
| ATGG | 120 |
| CTGG | 121 |
| GTGG | 122 |
| TTGG | 123 |
| ATAG | 124 |
| CTAG | 125 |
| GTAG | 126 |
| TTAG | 127 |
| ATCG | 128 |
| CTCG | 129 |
| GTCG | 130 |
| TTCG | 131 |
| ATGT | 132 |
| CTGT | 133 |
| GTGT | 134 |
| TTGT | 135 |
| ATAT | 136 |
| CTAT | 137 |
| GTAT | 138 |
| TTAT | 139 |
| ATCT | 140 |
| CTCT | 141 |
| GTCT | 142 |
| TTCT | 143 |
| AAGA | 144 |
| CAGA | 145 |
| GAGA | 146 |
| TAGA | 147 |
| AATA | 148 |
| CATA | 149 |
| GATA | 150 |
| TATA | 151 |
| AACA | 152 |
| CACA | 153 |
| GACA | 154 |
| TACA | 155 |
| AAGC | 156 |
| CAGC | 157 |
| GAGC | 158 |
| TAGC | 159 |
| AATC | 160 |
| CATC | 161 |
| GATC | 162 |
| TATC | 163 |
| AACC | 164 |
| CACC | 165 |
| GACC | 166 |
| TACC | 167 |
| AAGG | 168 |
| CAGG | 169 |
| GAGG | 170 |
| TAGG | 171 |
| AATG | 172 |
| CATG | 173 |
| GATG | 174 |
| TATG | 175 |
| AACG | 176 |
| CACG | 177 |
| GACG | 178 |
| TACG | 179 |
| AAGT | 180 |
| CAGT | 181 |
| GAGT | 182 |
| TAGT | 183 |
| AATT | 184 |
| CATT | 185 |
| GATT | 186 |
| TATT | 187 |
| AACT | 188 |
| CACT | 189 |
| GACT | 190 |
| TACT | 191 |

9. A system for storing data on deoxyribonucleic acid (“DNA”), the system comprising:
a receiver operable to receive one or more data files;
a processing element operable to:
segment the one or more data files into a plurality of data packets;
randomly select one or more packets from the plurality of data packets;
combine, using an algorithm, the selected one or more packets into an output;
attach a four-byte random seed to the output;
derive a sequence from the seeded output;
identify the sequence as a valid sequence or an invalid sequence; and
discard the sequence when the sequence is identified as an invalid sequence;
a DNA synthesizer operable to:
when the sequence is identified as a valid sequence, convert the sequence into a DNA quaternary sequence, said DNA quaternary sequence comprising two or more DNA bases;
synthesize the DNA sequence; and
store the DNA sequence.

10. The system of claim 9, wherein the algorithm is an exclusive or operation.

11. The system of claim 9, wherein the algorithm is a bitwise addition operation.

12. The system of claim 9, wherein the invalid sequence is a homopolymer.

13. The system of claim 9, wherein the invalid sequence comprises greater than a threshold number of duplicate bases.

14. The system of claim 9, wherein the two or more DNA bases include adenine, thymine, cytosine and guanine.

15. The system of claim 9, wherein the converting is based on a code table.

16. The system of claim 15 wherein the code table comprises the following code table:

| Quaternary | Decode |
|---|---|
| Code | Equivalent |
| ACGA | 0 |
| CCGA | 1 |
| GCGA | 2 |
| TCGA | 3 |
| ACTA | 4 |
| CCTA | 5 |
| GCTA | 6 |
| TCTA | 7 |
| ACAA | 8 |
| CCAA | 9 |
| GCAA | 10 |
| TCAA | 11 |
| ACGC | 12 |
| CCGC | 13 |
| GCGC | 14 |
| TCGC | 15 |
| ACTC | 16 |
| CCTC | 17 |
| GCTC | 18 |
| TCTC | 19 |
| ACAC | 20 |
| CCAC | 21 |
| GCAC | 22 |
| TCAC | 23 |
| ACTG | 24 |
| CCTG | 25 |
| GCTG | 26 |
| TCTG | 27 |
| ACAG | 28 |
| CCAG | 29 |
| GCAG | 30 |
| TCAG | 31 |
| ACGG | 32 |
| CCGG | 33 |
| GCGG | 34 |
| TCGG | 35 |
| ACGT | 36 |
| CCGT | 37 |
| GCGT | 38 |
| TCGT | 39 |
| ACTT | 40 |
| CCTT | 41 |
| GCTT | 42 |
| TCTT | 43 |
| ACAT | 44 |
| CCAT | 45 |
| GCAT | 46 |
| TCAT | 47 |
| AGTA | 48 |
| CGTA | 49 |
| GGTA | 50 |
| TGTA | 51 |
| AGAA | 52 |
| CGAA | 53 |
| GGAA | 54 |
| TGAA | 55 |
| AGCA | 56 |
| CGCA | 57 |
| GGCA | 58 |
| TGCA | 59 |
| AGTC | 60 |
| CGTC | 61 |
| GGTC | 62 |
| TGTC | 63 |
| AGAC | 64 |
| CGAC | 65 |
| GGAC | 66 |
| TGAC | 67 |
| AGCC | 68 |
| CGCC | 69 |
| GGCC | 70 |
| TGCC | 71 |
| AGTG | 72 |
| CGTG | 73 |
| GGTG | 74 |
| TGTG | 75 |
| AGAG | 76 |
| CGAG | 77 |
| GGAG | 78 |
| TGAG | 79 |
| AGCG | 80 |
| CGCG | 81 |
| GGCG | 82 |
| TGCG | 83 |
| AGTT | 84 |
| CGTT | 85 |
| GGTT | 86 |
| TGTT | 87 |
| AGAT | 88 |
| CGAT | 89 |
| GGAT | 90 |
| TGAT | 91 |
| AGCT | 92 |
| CGCT | 93 |
| GGCT | 94 |
| TGCT | 95 |
| ATGA | 96 |
| CTGA | 97 |
| GTGA | 98 |
| TTGA | 99 |
| ATAA | 100 |
| CTAA | 101 |
| GTAA | 102 |
| TTAA | 103 |
| ATCA | 104 |
| CTCA | 105 |
| GTCA | 106 |
| TTCA | 107 |
| ATGC | 108 |
| CTGC | 109 |
| GTGC | 110 |
| TTGC | 111 |
| ATAC | 112 |
| CTAC | 113 |
| GTAC | 114 |
| TTAC | 115 |
| ATCC | 116 |
| CTCC | 117 |
| GTCC | 118 |
| TTCC | 119 |
| ATGG | 120 |
| CTGG | 121 |
| GTGG | 122 |
| TTGG | 123 |
| ATAG | 124 |
| CTAG | 125 |
| GTAG | 126 |
| TTAG | 127 |
| ATCG | 128 |
| CTCG | 129 |
| GTCG | 130 |
| TTCG | 131 |
| ATGT | 132 |
| CTGT | 133 |
| GTGT | 134 |
| TTGT | 135 |
| ATAT | 136 |
| CTAT | 137 |
| GTAT | 138 |
| TTAT | 139 |
| ATCT | 140 |
| CTCT | 141 |
| GTCT | 142 |
| TTCT | 143 |
| AAGA | 144 |
| CAGA | 145 |
| GAGA | 146 |
| TAGA | 147 |
| AATA | 148 |
| CATA | 149 |
| GATA | 150 |
| TATA | 151 |
| AACA | 152 |
| CACA | 153 |
| GACA | 154 |
| TACA | 155 |
| AAGC | 156 |
| CAGC | 157 |
| GAGC | 158 |
| TAGC | 159 |
| AATC | 160 |
| CATC | 161 |
| GATC | 162 |
| TATC | 163 |
| AACC | 164 |
| CACC | 165 |
| GACC | 166 |
| TACC | 167 |
| AAGG | 168 |
| CAGG | 169 |
| GAGG | 170 |
| TAGG | 171 |
| AATG | 172 |
| CATG | 173 |
| GATG | 174 |
| TATG | 175 |
| AACG | 176 |
| CACG | 177 |
| GACG | 178 |
| TACG | 179 |
| AAGT | 180 |
| CAGT | 181 |
| GAGT | 182 |
| TAGT | 183 |
| AATT | 184 |
| CATT | 185 |
| GATT | 186 |
| TATT | 187 |
| AACT | 188 |
| CACT | 189 |
| GACT | 190 |
| TACT | 191 |
